(19) United States
(12) Patent Application Publication
(10) Pub. No.: US 2004/0253179 A1
(43) Pub. Date: Dec. 16, 2004

(54) Measuring gastrointestinal parameters

(75) Inventor: Wei-Kuo Chang, Taipei City (TW)

Correspondence Address: WHYTE HIRSCHBOECK DUDEK S C, 555 EAST WELLS STREET, SUITE 1900, MILWAUKEE, WI, 53202, US

(73) Assignee: ACM Medical Technologies, Inc., Kansas City, MO

(21) Appl. No.: 10/787,705

(22) Filed: Feb. 26, 2004

Related U.S. Application Data
(60) Provisional application No. 60/450,551, filed on Feb. 26, 2003.
(60) Provisional application No. 60/526,345, filed on Dec. 1, 2003.

(51) Int. Cl. A61K 49/00

(52) U.S. Cl. 424/9.1

(57) ABSTRACT

The present application relates to the methods of monitoring gastric residual volumes and dietary formula concentrations in gastric content samples using Brix value measurements as a means of evaluating feeding tolerance and gastric emptying in patients.

### PRIORITY CLAIM AND RELATED APPLICATION

The present application claims priority from U.S. Provisional Application entitled “Monitoring Gastric Residual Volume and Formula Concentration”, Ser. No. 60/450,551, filed Feb. 26, 2003 and U.S. Provisional Application entitled “Measuring Gastrointestinal Parameters”, Ser. No. 60/526,345, filed Dec. 1, 2003, which are incorporated herein by reference in their entirety for all purposes.

### FIELD

The present application relates to the methods of monitoring gastric emptying and evaluating feeding tolerance in patients receiving enteral nutrition.

### BACKGROUND

Enteral nutrition is generally preferred over parenteral nutrition because of its lower cost, lower rate of complications, and effective preservation of gut structure and function. Many critically ill patients cannot tolerate nasogastric tube feeding, developing manifestations of intolerance including nausea, vomiting, abdominal distension, and aspiration. Gastric residual volumes are widely used to evaluate feeding tolerance and gastric emptying. High gastric residual volumes raise concern about intolerance to gastric feeding and the potential risk for regurgitation and aspiration pneumonia. Values of gastric residual volumes cited as being high in patients receiving nasogastric feeding typically range from 75 to 500 ml. However, controversy exists regarding the accuracy of these measurements.

Conventional use of gastric residual volume obtained by aspiration via a syringe is often inaccurate and unreliable in measuring true volume of contents present in the stomach at any given time. Although gastric residual volumes obtained by aspiration from a nasogastric feeding tube (Asp GRVs) are widely used to evaluate tolerance and gastric emptying of enteral feedings, several reports have shown that Asp GRVs by themselves are poorly correlated with gastric emptying, incidence of regurgitation, and risk of pulmonary aspiration. The conventional practice of calculating gastric residual volume typically does not take into account the fact that fluids accumulating in the stomach of the patient during nasogastric tube feeding often include not only the tube feeding formula itself, but also swallowed saliva and gastric secretions. Therefore, gastric residual volumes alone cannot distinguish the additional volume of endogenous secretions in a patient who is effectively emptying the volume of exogenous feeding. As a result, use of aspirated gastric residual volumes as a monitor for gastric emptying is currently limited by poor sensitivity and an inability to aspirate the complete volume of gastric contents. The sensitivity of Asp GRVs for detecting aspiration through a range of designated threshold values is only 1.9-8.1%. Moreover, conventional calculation of aspirated gastric residual volumes is imprecise and cannot distinguish the components of retained enteral formula from the large volume of naturally occurring endogenous secretions.

The true gastric residual volume is determined by the dynamic balance between input (e.g., infused formula and endogenous secretions) and output (e.g., gastric emptying of the stomach). Typically, endogenous secretions contribute 1500 ml of salivary secretions and 3000 ml of gastric juice per day. Therefore, an accurate method to determine total volume of contents in the stomach and a system to differentiate the exact volume of the component of formula in that mixed solution of gastric contents would be helpful in evaluating treatment in patients receiving gastric feeding. Recent evidence suggests that conventional calculation of Asp GRVs (especially when performed by syringe aspiration) does not adequately measure true volume of gastric contents remaining in the stomach of Asp because it cannot differentiate the components of the gastric contents (infused formula versus endogenous secretions).

Refractometry is a useful technique because of its minimal expense and ease of utilization. The Brix value is a measurement, typically measured using refractometry, of total soluble solids in solution. This value is a constant for a pure substance under standard conditions of temperature and pressure. The Brix value closely correlates with the molar fractions of the components. In other words, the overall Brix value of a mixed solution approximates the additive sum of the Brix values of its individual components. Brix values have been used in a number of clinical settings to determine the concentration of mixed substances such as drugs, food, fruit juices, and parenteral nutrition solutions. However, there is not much known about Brix values related to enteral nutrition solutions or its correlation to gastric emptying. For example, it is currently unknown what nutrients in dietary formula are necessary for Brix value determination, what parameters affect Brix value measurements of gastric contents or the correlation between Brix values and gastric content components.

### SUMMARY

A method of monitoring feeding tolerance in a patient receiving enteral nutrition is described and involves infusing dietary formula into the stomach of a patient, measuring the aspirated gastric residual volume of the infused patient, evaluating the aspirated gastric residual volume, whereby a lower gastric residual volume value indicates acceptable feeding tolerance and a higher gastric residual volume value indicates additional monitoring, calculating a Brix value ratio obtained by a Brix value dilution test, evaluating the Brix value ratio, whereby a ratio of less than approximately 70% indicates acceptable feeding tolerance and a ratio above approximately 70% indicates additional monitoring, calculating the volume of formula remaining in the stomach, and evaluating the volume of formula remaining in the stomach, whereby a formula volume approximately less than the infusion rate indicates acceptable feeding tolerance and a formula volume greater than the infusion rate indicates additional monitoring.

A method of determining the concentration of dietary formula is also disclosed, involving infusing dietary formula to the stomach of a subject, measuring a Brix value of the infused dietary formula, determining a slope value derived from Brix values of serially diluted dietary formula over a determined concentration range, and dividing the Brix value of the infused dietary formula by the slope value.

A method is also provided for determining gastric residual volume by measuring the Brix value of a gastric content sample, adding a known volume of water to the gastric content sample to form a post-dilution sample, infusing the post-dilution sample from the stomach, measuring the Brix value of the aspirated post-dilution sample, and multiplying the Brix value of the gastric content sample by the known volume of water and dividing the resulting product by the difference between the pre-dilution Brix value and post-dilution Brix value.

Also methods of monitoring gastric content emptying and feeding tolerance in a patient receiving dietary formula, and methods of determining the volume of dietary formula volume remaining in the stomach and the volume of gastric juice in stomach are also disclosed.

These and other features will be appreciated from review of the following detailed description along with the accompanying figures.

### BRIEF DESCRIPTION OF THE DRAWINGS

FIG. 1 shows a representative determination of dietary formula concentration;

FIG. 2 shows a representative determination of gastric residual volume;

FIG. 3 shows a representative example of determining the volume of dietary formula remaining in stomach;

FIG. 4 shows a method of monitoring feeding tolerance in a patient receiving enteral nutrition using Brix value measurements in conjunction with conventional aspirated gastric residual volume measurements; and

FIG. 5 shows an experimental derivation of calculating the gastric residual volume using Brix value measurements.

### DETAILED DESCRIPTION

This present application describes the monitoring of gastric tolerance and gastric emptying through measurement of Brix values gastric contents.

Method of Measuring the Concentration of Dietary Formula

Disclosed is a method of measuring the percent concentration of dietary formula by infusing dietary formula into the stomach of a subject. Infusing may be accomplished by nasogastric feeding, enteral nutrition feed, or any known tube feeding technique. Any dietary formula can be used with this method. For example, in one embodiment, the dietary formula is a dietary formula for gastric tube feeding or any type of enteral nutrition. In another embodiment, the dietary formula is liquid dietary formula or polymeric diet formula. Likewise, any ranges of the concentration of dietary formula can be monitored by this method. That is, 0-100% of the dietary formula can be monitored by refractometry. For example, full-strength polymeric diet would be considered 100% concentration dietary formula.

The Brix value of the infused dietary formula is measured. In one embodiment, the Brix value is measured with a refractometer. However, other known methods of obtaining a Brix value may also be used.

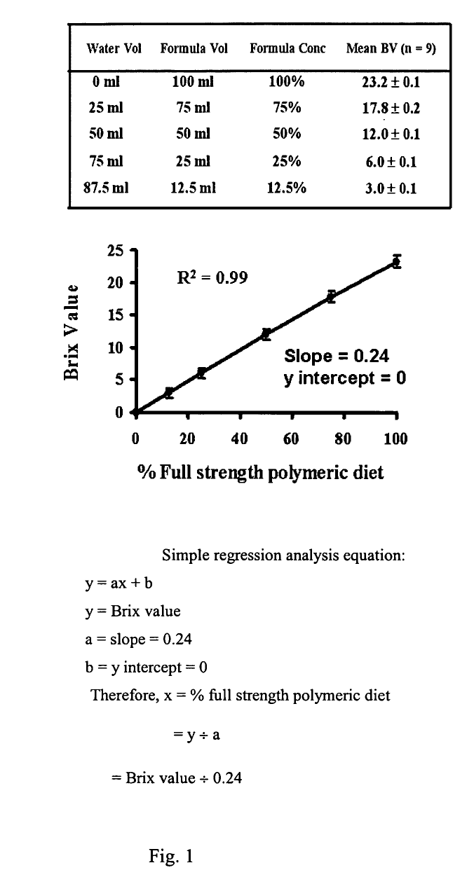

A slope value is derived from Brix values of serially diluted dietary formula over a determined concentration range. One embodiment is shown in FIG. 1. In this non-limiting example, the Brix values of an infused dietary formula are plotted against serial dilutions of that formula exhibited as concentration percentages of full strength (100%) dietary formula, or polymeric diet. The slope value is calculated according to known regression analysis methods. In some embodiments, the dietary formula may be diluted by distilled water, gastric juice, saliva, NaCl or dextrose solutions. However, other known diluents may also be used. In one embodiment, the slope value is 0.24. The percent concentration of dietary formula may be expressed as:

$$\% conc . dietary formula = \frac{Brix value infused dietary formula}{slope value}$$

The Brix values of polymeric dietary formula have a linear additive relationship with the dietary formula concentration (R²=0.99), which is exemplified in FIG. 1. With such a high degree of correlation, the measured Brix value may be correlated to the percent concentration of the formula (e.g., % full strength polymeric diet) at any dilution.

In other embodiments, the percent concentration of dietary formula in the gastric content is not affected by the body secretions, pH value, temperature or the residence time of the dietary formula in stomach. These and other features are further described in non-limiting detail through the examples provided herein.

Method of Determining the Gastric Residual Volume

A method of determining gastric residual volume is also disclosed. The Brix value of a gastric content sample is measured to obtain a pre-dilution Brix value, BV1. The Brix value may be measured with a refractometer or any other device or technique known in the art. The gastric content sample may, for example, contain dietary formula, bodily secretions (e.g., gastric juices or saliva), and mixtures thereof. Of course, other components known in the art may also be contained in the gastric content sample. The Brix value may be measured from a gastric content sample obtained in vitro or after the sample has been aspirated from the stomach of a patient.

A known volume of water sample is added to the gastric content sample to form a post-dilution sample. The amount of water added to the sample may be easily determined by one skilled in the art, depending on the size of the sample, the dietary formula, the condition of the subject, etc. In the case of a sample obtained in vivo, the water is added after the sample has been aspirated from the stomach of the patient. In some embodiments, the volume of water ranges between 10 ml to 500 ml. The gastric content is infused into the stomach using known infusion methods. Upon aspirating the resulting post-dilution sample from the stomach, the Brix value of the aspirated post-dilution sample is measured to obtain a value BV2. Calculation of the gastric residual volume may be expressed as:

$$\frac{BV1 \times added volume of water}{( BV1 - BV2 )}$$

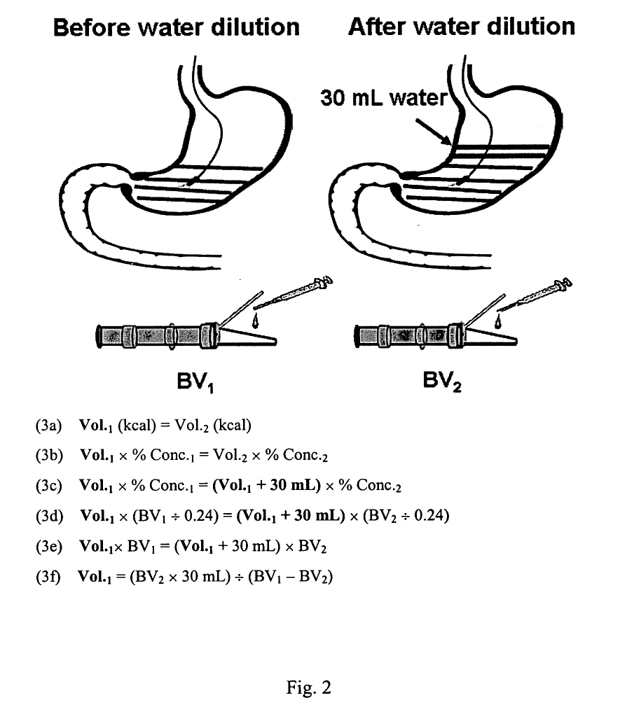

A representative determination of the gastric residual volume is exemplified in FIG. 2. Before water dilution, the calculated gastric residual volume was set as Vol. 1, % full strength polymeric dietary as % Conc. 1, and the Brix value of gastric contents as BV1. After 30 ml water dilution in the stomach, the gastric volume was set as Vol. 2, % full strength polymeric dietary as % Conc. 2, and the Brix value of gastric content as BV2. An added dilutional 30 ml volume of distilled water was infused via nasogastric tube. The stomach contents again were thoroughly mixed (using a 60-ml syringe, filled and emptied 3 times), and a 2 ml sample was obtained for a final Brix value measurement. The gastric residual volume (Vol. 1) was calculated according to the equation shown in FIG. 2.

Method of Determining the Amount of Dietary Formula Volume Remaining and Gastric Juice Volume in Stomach

A method of determining the amount of dietary formula volume remaining in the stomach involves determining the gastric residual volume of a subject and multiplying this volume by the concentration of dietary formula. In some embodiments, this concentration may be expressed as the percentage full strength of dietary formula or polymeric diet.

The calculated gastric residual volume may be obtained from measuring the gastric residual volume. Any dietary formula can be used with this method. In one embodiment, the dietary formula is the dietary formula for gastric tube feeding or any type of enteral nutrition. In another embodiment, the dietary formula is polymeric diet formula or liquid dietary formula. However, other known dietary formulas may also be used. Typically, a refractometer is used to measure the Brix values, but other known methods may also be used.

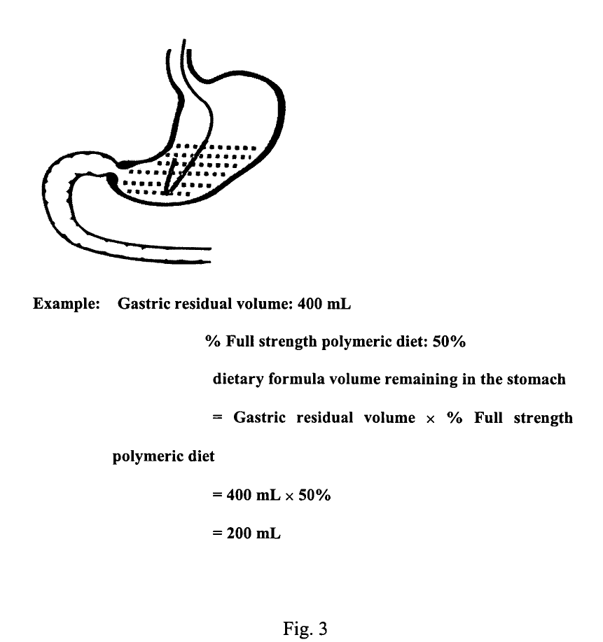

One embodiment of determining the amount of dietary formula remaining in the stomach is shown in FIG. 3. In this example, the gastric residual volume was measured or calculated at 400 ml. The dietary formula infused into the patient was 50% full strength polymeric diet formula. Thus, the volume of dietary formula remaining in the stomach at the point in time that the gastric residual volume was measured or calculated, is 200 ml.

A method of measuring gastric juice volume in stomach is also disclosed by measuring the dietary formula volume remaining in the stomach of a subject, calculating or measuring the gastric residual volume, and determining the value derived from the difference between the gastric residual volume and the dietary formula remaining in the stomach. The dietary formula volume remaining in the stomach can be obtained, for example, as disclosed above. However, other known methods of calculating dietary formula volume may also be used.

Based on the disclosed methods and the representative examples provided throughout, dietary ranges of nutrients (such as carbohydrate, protein, and fat) may be monitored by refractometry. The Brix value is a linear additive function of the concentration of nutrients present in a solution and Brix values correlate with the concentrations of dietary formula independent of pH, temperature, and the types of solutions. Thus, the Brix value can be used clinically and in research to monitor dietary formula concentrations, and therefore be used in clinical practice to evaluate dietary formula during storage, preparation, and administration.

The Brix value measurement of gastric contents can be used to monitor both gastric residual volume and food content in patients receiving enteral nutrition, e.g., by nasogastric feeding. The Brix value measurement of gastric juice can be used to monitor gastric emptying in patients receiving nasogastric feeding, thereby providing additional information to calculation of the aspirated gastric residual volume.

Because the Brix value measurements for various dilutions of the polymeric dietary have minimal variability in vitro, the disclosed methods permits bedside measurements with a high degree of reproducibility. In another application, saliva and gastric secretions have few dissolved substances and therefore have correspondingly low Brix value, close to that distilled water. Therefore, the disclosed methods can also be used to determine whether gastric contents are comprised predominately of dietary formula or digestive secretions in patients receiving polymeric dietary feeding.

Brix value measurement of gastric contents can be used in clinical practice, complementing the use and interpretation of gastric residual volumes, particularly for management of patients receiving nasogastric tube feeding. Gastric residual volume is determined by the dynamic balance between input (endogenous saliva plus gastric juice and exogenous formula) and output (gastric emptying) from the stomach. A low Brix value (especially if there is a steady decrease following bolus infusion) indicates a low concentration of formula remaining and implies adequate emptying of formula from the stomach. This may be confirmed using the dilution technique and the change in Brix value to determine calculated gastric residual volume and specific volume of formula remaining.

A method of monitoring gastric content emptying and tolerance in patients receiving dietary formula is further disclosed. The Brix value ratio, an alternative measure of the amount of dietary formula retained in the stomach, may be calculated by:

$$\frac{post \text{-} dilution Brix value}{pre \text{-} dilution Brix value}$$

The pre-dilution Brix value may be obtained from measuring the Brix value of a sample aspirated from the gastric contents of the stomach of a patient infused with dietary formula. After a known volume of water has been added to the gastric contents and reinstalled in the patient, the post-dilution Brix value may be obtained from reaspirating a sample and measuring the Brix value.

The specific component of gastric residual volume that is volume comprised of dietary formula may be determined using the equation:

$$Volume of dietary formula remaining in stomach = \frac{calculated gastric residual volume \times \frac{( pre \text{-} dilution Brix values )}{0.24}}{100}$$

To assist in understanding the present application, the following examples are included and describe the results of a series of experiments. The following examples relating to this application should not be construed to specifically limit the application or such variations of the application, now known or later developed, which fall within the scope of the application as described and claimed herein.

### EXAMPLES

### Example 1

### Use of Brix Value to Monitor Dietary Formula Concentration

Materials and Methods

Brix values for nutrients such as minerals, vitamins mixtures, carbohydrate, protein, fat, and polymeric dietary were determined with a refractometer (N.O.W 507-1, Nippon Optical Works; Tokyo, Japan). A solution of minerals (Ringer's solution) was obtained from YF Chemical Corporation (Taipei, Taiwan), and consisted of sodium chloride (8.6 mg/ml), potassium chloride (0.3 mg/ml), and calcium chloride (0.33 mg/ml). Vitamins (Lyo-povigen, a parenteral vitamin mixture) was also obtained from YF Chemical Corporation (Taiwan), and contained vitamin A palmitate (12 IU/ml), vitamin D2 (1 IU/ml), vitamin E (0.005 IU/ml), vitamin C (0.5 mg/ml), vitamin B1 (0.05 mg/ml), vitamin B2 (0.01 mg/ml), vitamin B6 (0.015 mg/ml), niacinamide (0.1 mg/ml), and d-panthenol (0.025 mg/ml). Carbohydrate (Carb-aid, Corn starch) and protein (Whey-aid, lactoalbumin) were purchased from Nutritec-Enjoy Nutrition Center, Taiwan. Fat (Intralipid) was purchased from Frenius Kabi AB, Uppsala, Sweden. Full strength polymeric dietary (Osmolite HN, Ross, Ohio, USA) contained carbohydrate (17 g/100 ml), protein (5.3 g/100 ml), and fat (4.1 g/100 ml).

The Brix values were measured using a hand-held refractometer, whose Brix scale (% Brix) of 0-32 could be read in 0.2 increments. To measure the solute concentration, one or two drops of the specimen fluid were placed in a designated window. The refractometer was calibrated with distilled water before each measurement.

Statistical Analysis

Results are presented as the mean ±SEM. Correlation coefficients following linear regression analysis were used to evaluate the relationship between Brix values and dietary formula concentrations. Differences were considered statistically significant when P<0.05.

Results

Brix Values of Nutrients

The Brix values of nutrients and dietary formula are listed in Table 1. Distilled water, minerals, and vitamins contained little dissolved material and had correspondingly low Brix values of 0±0, 1.2±0.1, and 0.4±0.1, respectively. Carbohydrate (17 g/100 ml), protein (5.3 g/100 ml), fat (4.1 g/100 ml), and full-strength polymeric dietary had high concentrations of dissolved nutrients and correspondingly high Brix values of 12.1±0.6, 6.5±0.1, 6.0±0. 1, and 23.5±0.1, respectively.

TABLE 1
Brix values of nutrients

| Nutrient | Brix value (% Brix) |
|---|---|
| Distilled water | 0 ± 0 |
| Minerals (Ringer's solution) | 1.2 ± 0.1 |
| Vitamins (parenteral vitamin mixture) | 0.4 ± 0.1 |
| Carbohydrate (17 g/100 ml) | 12.1 ± 0.6 |
| Protein (5.3 g/100 ml) | 6.5 ± 0.1 |
| Fat (4.1 g/100 ml) | 6.0 ± 0.1 |
| Polymeric diet (full-strength) | 23.5 ± 0.1 |

Results presented are mean ± SD. Polymeric diet (Osmolite HN).

Molar Refractivities in Solution of Mixed Nutrients Were Additive. Table 2 shows Brix values of pure nutrients (such as carbohydrate, protein, and fat) and mixtures of these. Three dilutions (50%, 100% and 200% of the starting concentration) of carbohydrate, protein, and fat were made with distilled water. The Brix value was a linear additive function of the solute concentration, regardless of whether the solute was carbohydrate, protein, or fat.

TABLE 2
Brix values of pure and mixed nutrients

| Nutrient | Brix value (% Brix) |
|---|---|
| Carbohydrate (8.5 g/100 ml) | 6.8 ± 0.6 |
| Carbohydrate (17 g/100 ml) | 12.4 ± 0.6 |
| Carbohydrate (34 g/100 ml) | 25.8 ± 0.6 |
| Protein (2.6 g/100 ml) | 3.1 ± 0.1 |
| Protein (5.3 g/100 ml) | 6.3 ± 0.1 |
| Protein (10.5 g/100 ml) | 11.4 ± 0.1 |
| Fat (2.1 g/100 ml) | 3.1 ± 0.1 |
| Fat (4.1 g/100 ml) | 6.0 ± 0.1 |
| Fat (8.2 g/100 ml) | 11.9 ± 0.1 |
| Carbohydrate^{a} + protein^{b} | 19.2 ± 0.1 |
| Carbohydrate^{a} + protein^{b} + fat^{c} | 23.0 ± 0.1 |

^{a}Carbohydrate: 17 g/100 ml.
^{b}Protein: 5.3 g/100 ml.
^{c}Fat: 4.1 g/100 ml.
Results presented are mean ± SD.

For nutrient solutions, consisting of some combination of carbohydrate (17 g/100 ml), protein (5.3 g/100 ml), and fat (4.1 g/100 ml), Brix value was also an additive function of the component concentration. For example, a mixture of carbohydrate (17 g/100 ml) and protein (5.3 g/100 ml) had a Brix value of 19.2±0.1, and a mixture of carbohydrate (17 g/100 ml), protein (5.3 g/100 ml), and fat (4.1 g/100 ml) had a Brix value of 23.0±0.1, which was close to the Brix value (23.5±0.1) of full-strength polymeric diet.

Brix values of nutrients at various pHs and temperatures. Table 3 shows the effect of pH on the Brix value of serially diluted polymeric dietary formula. Brix values correlated with the polymeric dietary formula concentration at each pH (P<0.001). However, the Brix values of the polymeric dietary formula were lower at pH 1 and 4 than at pH 7 and 8. The decrease of Brix value may be due to the protein denaturation that occurs in very acid solutions. Table 4 shows the effect of temperature on the Brix values of serially diluted polymeric dietary formula. The Brix values correlated with the concentrations of dietary formula at all temperatures investigated (P<0.001).

TABLE 3
Brix values of polymeric diet at various pH values

| | % Full-strength polymeric diet | | | | | | |
|---|---|---|---|---|---|---|---|
| pH | 0 | 12.5 | 25 | 50 | 75 | 100 | R² |
| pH 1 | 0 ± 0 | 3.2 ± 0.3 | 3.0 ± 0.1 | 9.6 ± 0.3 | 13.1 ± 0.3 | 17.2 ± 0.1 | 0.93 |
| pH 4 | 0 ± 0 | 2.1 ± 0.1 | 4.2 ± 0.2 | 9.0 ± 1.0 | 12.4 ± 0.3 | 16.3 ± 0.1 | 0.93 |
| pH 7 | 0 ± 0 | 3.0 ± 0.1 | 6.1 ± 0.1 | 12.3 ± 0.1 | 17.9 ± 0.1 | 23.3 ± 0.1 | 1.00 |
| pH 8 | 0 ± 0 | 3.0 ± 0.1 | 6.1 ± 0.1 | 12.3 ± 0.1 | 17.3 ± 0.2 | 23.4 ± 0.1 | 0.99 |

Brix values (% Brix) of polymeric diet (Osmolite HN) diluted in distilled water and pH adjusted to different values. Result are presented as means ± SEM.

TABLE 4
Brix values of polymeric diet at various temperatures

| | % Full-strength polymeric diet | | | | | | |
|---|---|---|---|---|---|---|---|
| Temp | 0 | 12.5 | 25 | 50 | 75 | 100 | R² |
| 4° C. | 0 ± 0 | 3.2 ± 0.1 | 6.1 ± 0.2 | 12.0 ± 0.1 | 17.3 ± 0.4 | 23.4 ± 0.1 | 0.99 |
| 23° C. | 0 ± 0 | 3.0 ± 0.1 | 6.0 ± 0.1 | 12.0 ± 0.2 | 17.3 ± 0.2 | 23.2 ± 0.1 | 0.99 |
| 37° C. | 0 ± 0 | 3.0 ± 0.1 | 6.1 ± 0.1 | 12.1 ± 0.1 | 16.3 ± 0.1 | 23.5 ± 0.3 | 0.99 |

Brix values (% Brix of polymeric diet (Osmolite HN) diluted in distilled water $$ to different temperatures. Results are presented as means ± SEM.

Brix values of polymeric dietary in fasting gastric juice. Table 5 shows the effect of gastric juice dilution on the Brix value of the polymeric dietary formula. The Brix value measurements of polymeric dietary in fasting gastric juice were made at 5, 30, 120 and 240 min, respectively. Brix values correlated with the concentration of polymeric dietary diluted in fasting gastric juice at each time with minimal variability (R²<0.98). Therefore, polymeric dietary formula concentration in gastric contents can be estimated on the basis of the linear regression equation: Full strength polymeric diet % concentration=Brix value÷0.24, wherein 0.24 is the slope of serially diluted dietary formula concentration. For example, a Brix value of 6.0 and 12 in gastric contents corresponds to a 25% and 50% full-strength polymeric dietary concentration, expressed as % concentration of dietary formula.

TABLE 5
Brix values of polymeric diet in gastric juice

| | % Full-strength polymeric diet | | | | | | |
|---|---|---|---|---|---|---|---|
| Time (min) | 0 | 12.5 | 25 | 50 | 75 | 100 | R² |
| 5 | 2.0 ± 0.4 | 4.6 ± 0.3 | 6.8 ± 0.4 | 12.3 ± 0.8 | 18.0 ± 0.2 | 23.2 ± 0.4 | 0.99 |
| 30 | 2.1 ± 0.4 | 4.6 ± 0.4 | 6.8 ± 0.5 | 12.2 ± 0.7 | 18.0 ± 0.2 | 23.3 ± 0.1 | 0.98 |
| 120 | 2.0 ± 0.4 | 4.7 ± 0.3 | 6.9 ± 0.6 | 12.0 ± 0.7 | 17.8 ± 0.5 | 23.2 ± 0.1 | 0.98 |
| 240 | 2.0 ± 0.4 | 4.8 ± 0.5 | 7.0 ± 0.6 | 12.0 ± 0.8 | 17.6 ± 0.7 | 23.2 ± 0.1 | 0.98 |

Brix values (% Brix) of polymeric diet (Osmolite HN) diluted in fasting gastric juice. Results are presented as means ± SEM (n = 6).

### Example 2

### Monitoring Bolus Nasogastric Feeding by the Brix Value Determination of Gastric Contents and Residual Volume Measurement of Gastric Contents

Materials and Methods

All BV measurements were made using a hand-held refractometer (Model N.O.W. 507-1, Nippon Optical Works, Tokyo, Japan), whose Brix scale of 0-32 could be read in 0.2 increments. The refractometer was calibrated with distilled water before each measurement. One or two drops of the specimen fluid were placed on a designated window for observation, all measurements made at room temperature using natural light. In this way, the concentration of soluble solids in solution was measured at the bedside for each specimen.

Brix values for a polymeric (Osmolite HN, Abbott Laboratories, Columbus, Ohio) and five solutions (distilled water, 0.9% sodium chloride, 5% dextrose, fasting saliva, and gastric juice) were determined with the refractometer. Each liquid was evaluated six times. Serial dilutions of the polymeric formula (100%, 50%, 25%, 12.5%, 6.2%, and 0%) were made with three of the solutions (distilled water, saliva, and gastric juice) and is shown in the table below. The Brix value was measured in vitro, again performing six separate evaluations for each dilution of the polymeric formula.

The Brix Values for the polymeric diet diluted with different solutions.

| | % Full strength of polymeric diet (Osmolite HN) | | | | | |
|---|---|---|---|---|---|---|
| Solution | 0 | 6.2 | 12.5 | 25 | 50 | 100 |
| Distilled water | 0 ± 0 | 2.1 ± 0.4 | 3.5 ± 0.8 | 6.0 ± 0.9 | 12.2 ± 0.8 | 23.2 ± 0.3 |
| Saliva | 1.3 ± 0.4 | 2.2 ± 0.6 | 4.4 ± 0.6 | 7.3 ± 0.7 | 12.4 ± 0.9 | 23.2 ± 0.5 |
| Gastric juice | 1.9 ± 0.6 | 4.4 ± 1.4 | 5.4 ± 1.3 | 7.8 ± 1.4 | 13.3 ± 1.2 | 23.2 ± 0.4 |

Results are presented as, mean values for six separate determinations ± SEM.

Patients receiving bolus nasogastric feeding were used in this study. All subjects were fed the full strength polymeric dietary via a 14 French nasogastric feeding tube. The polymeric dietary composition was 16.7% protein, 54.3% carbohydrate, and 29.0% lipid. Caloric requirements were calculated using the Harris-Benedict equation. A total of 250 ml of the polymeric dietary was administered by bolus infusion every 3-6 hours in the 24 hours prior to the study. Aspirated gastric residual volumes were obtained before each bolus feed by aspiration of the feeding tube. Aspirated gastric residual volumes were obtained first in the supine position, and then in the right lateral decubitus position. Following this preliminary period of monitoring, patients were arbitrarily divided into two groups based on conventional use of aspirated gastric residual volume; patients with low gastric residual volumes (<75 ml) were placed in group 1, and patients with higher gastric residual volumes (>75 ml on at least two occasions) were placed in group 2. In total, there were 25 subjects in group 1 (age: range=59-84, mean±SD=75.9±6.6 years) and 18 patients in group 2 (age: range=44-79, mean±SD=70.6±10.6 years).

After overnight fasting, all remaining gastric juice was aspirated from the stomach via the nasogastric tube using a 60 ml syringe. Then, all subjects received bolus infusion of 250 ml of the polymeric diet. Immediately after feeding, an attempt was made to thoroughly mix the food content in the stomach by aspirating and reinfusing of the nasogastric tube three times with a 60 ml syringe.

Sequential Brix value determinations were made on 2 ml samples of gastric contents at 0, 30, 60, 120, and 180 minutes intervals. At 180 minutes, any fluid remaining in the stomach was aspirated. Its volume was recorded as the aspirate gastric residual volume (Asp GRV), the Brix value measurement made, (pre-diluted BV) and then the contents were reinstilled into the stomach. An added dilutional 30 ml volume of distilled water was infused via the nasogastric tube. The stomach contents again were thoroughly mixed, and a 2 ml sample was obtained for a final BV measurement (Post Dilute BV). The Calculated GRV was determined using the equation: Calculated GRV×PreDilute BV (Calculated GRV+30 ml)×PostDilute BV. Specific volume of formula remaining at 180 minutes was defined by 2 equations; % Concentration=BV_{180 min}/0.24 and Volume_{formula}=% Concentration×Calculated GRV.

The calculated GRV was determined by the equation:

$$\frac{Calc . GRV = 30 mi . Pre \text{-} Diluted BV}{Pre \text{-} Diluted B - Post \text{-} Diluted BV}$$

Results

Results are presented as mean values±SEM. The Student's t test was used to assess differences in results between patients in group 1 and group 2. A p value of less than 0.05 was considered to be statistically significant.

Immediately following the 250 mL polymeric dietary bolus feeding, the mean Brix values, (BVs), of the gastric contents in vivo were shown to be lower both in group 1 and group 2 (19.6±1.0 versus 20.3±1.1, respectively) than the mean values for the polymeric formula made in vitro (23.2±0.3). This decrease in the Brix value most likely was due to the dilutional effect of endogenous gastric juices present in the stomach at the time of feeding.

The serial changes in Brix values for gastric content for the two groups following the bolus polymeric dietary feeding are shown in Tables 6 and 7. Mean serial Brix value measurements decreased in both groups after bolus feeding. For patients in group 2, the decrease was less, such that at 180 minutes patients in group 2 had a significantly higher mean Brix value for gastric contents than those patients in group 1 (10.1±0.7 versus 5.1±0.9, respectively, p<0.01). Tables 6 and 7 also show that the aspirated gastric residual volume at 180 minutes was significantly higher for patients in group 2 than for those in group 1 (72±12 versus 18±5 mL, respectively, p<0.01).

TABLE 6
Serial Brix values and gastric residual volumes (GRVs) for patients in group 1 (low GRVs)

| | | | | | | Residual volume (ml) | | |
|---|---|---|---|---|---|---|---|---|
| Patient | Brix value of gastric juice | | | | | Aspirated | Calculated | Volume |
| no. | 0 min | 30 min | 60 min | 120 min | 180 min | GRV | GRV | Formula |
| 1 | 13.9 | 12.2 | 5.3 | 8.5 | 9.6 | 30 | 23 | 9 |
| 2 | 22.3 | 14.3 | 14.3 | 14.3 | 14.8 | 0 | 3 | 2 |
| 3 | 23.4 | 19.5 | 13.7 | 10.3 | 11.6 | 70 | 115 | 56 |
| 4 | 22.6 | 14.6 | 13.2 | 12.4 | 10.1 | 10 | 31 | 13 |
| 5 | 11.9 | 10.4 | 8.2 | 4.6 | 4.9 | 10 | 215 | 44 |
| 6 | 21.5 | 13.5 | 12.3 | 9.4 | 8.5 | 30 | — | — |
| 7 | 22.4 | 13.2 | 12.5 | 7.2 | 8.7 | 20 | — | — |
| 8 | 23.3 | 20.4 | 14.3 | 10.3 | 9.1 | 79 | 68 | 26 |
| 9 | 14.5 | 11.3 | 9.4 | 5.4 | 5.3 | 32 | 92 | 20 |
| 10 | 12.9 | 10.6 | 8.3 | 5.3 | 4.3 | 12 | 185 | 33 |
| 11 | 13.3 | 9.2 | 8.1 | 5.0 | 4.2 | 10 | — | — |
| 12 | 14.8 | 10.3 | 8.3 | 6.4 | 4.8 | 15 | — | — |
| 13 | 17.6 | 12.3 | 10.4 | 7.4 | 4.6 | 70 | 62 | 12 |
| 14 | 17.7 | 12.4 | 9. | 7.5 | 4.4 | 78 | 80 | 15 |
| 15 | 14.8 | 10.3 | 8.3 | 6.4 | 4.8 | 15 | — | — |
| 16 | 20.2 | 18.4 | 15.3 | 4.6 | 3.5 | 8 | 45 | 7 |
| 17 | 17.2 | 14.5 | 10.2 | 4.4 | 2.6 | 32 | — | — |
| 18 | 16.5 | 12.5 | 9.3 | 3.6 | 2.5 | 30 | — | — |
| 19 | 16.4 | 12.4 | 9.3 | 3.5 | 2.3 | 5 | — | — |
| 20 | 22.3 | 14.4 | 10.0 | 5.4 | 2.9 | 9 | 15 | 2 |
| 21 | 22.3 | 21.2 | 20.5 | 3.3 | 2.7 | 1 | — | — |
| 22 | 20.9 | 17.4 | 14.3 | 3.7 | 2.3 | 5 | 47 | 4 |
| 23 | 26.2 | 16.3 | 14.1 | 12.3 | 2.0 | 8 | 30 | 3 |
| 24 | 23.8 | 12.7 | 10.6 | 4.3 | 1.5 | 0 | 15 | 1 |
| 25 | 26.0 | 15.6 | 15.2 | 13.4 | 1.3 | 0 | 48 | 3 |
| Mean ± SEM | 19.6 ± 1.0 | 14.0 ± 0.8 | 11.4 ± 0.8 | 7.1 ± 0.8 | 5.1 ± 0.9* | 18 ± 5* | 67 ± 15* | 6 ± 2* |

(*p < 0.05 for mean values group 1 versus group 2)

TABLE 7
Serial Brix values and gastric residual volumes (GRVs) for patients in group 2 (higher GRVs)

| | | | | | | Residual volume (ml) | | |
|---|---|---|---|---|---|---|---|---|
| Patient | Brix value of gastric juice | | | | | Aspirated | Calculated | Volume |
| no. | 0 min | 30 min | 60 min | 120 min | 180 min | GRV | GRV | Formula |
| 1 | 16.4 | 14.5 | 11.2 | 11.3 | 12.3 | 15 | 89 | 46 |
| 2 | 13.4 | 12.4 | 12.4 | 8.5 | 9.3 | 30 | 26 | 10 |
| 3 | 17.8 | 14.1 | 12.4 | 13.4 | 12.3 | 35 | 138 | 71 |
| 4 | 22.1 | 19.2 | 16.3 | 14.4 | 14.4 | 190 | 197 | 118 |
| 5 | 19.2 | 18.2 | 16.2 | 15.1 | 11.2 | 37 | 82 | 38 |
| 6 | 23.3 | 19.2 | 16.5 | 15.5 | 13.0 | 210 | 165 | 89 |
| 7 | 20.2 | 16.6 | 18.2 | 15.3 | 10.6 | 47 | 215 | 95 |
| 8 | 29.1 | 25.0 | 22.1 | 15.5 | 14.2 | 65 | 79 | 47 |
| 9 | 29.0 | 24.5 | 20.3 | 14.7 | 14.2 | 55 | 55 | 33 |
| 10 | 23.1 | 19.3 | 13.3 | 10.2 | 11.4 | 70 | 125 | 60 |
| 11 | 23.3 | 17.2 | 14.4 | 12.3 | 11.2 | 74 | 104 | 49 |
| 12 | 12.5 | 9.3 | 7.7 | 6.2 | 5.5 | 65 | 108 | 25 |
| 13 | 21.2 | 17.4 | 13.7 | 6.3 | 8.6 | 60 | 93 | 33 |
| 14 | 21.5 | 18.4 | 14.6 | 10.2 | 8.2 | 70 | 68 | 23 |
| 15 | 14.2 | 12.5 | 8.8 | 6.7 | 5.1 | 75 | — | — |
| 16 | 22.2 | 18.5 | 12.2 | 8.6 | 5.3 | 85 | — | — |
| 17 | 15.2 | 14.2 | 12.2 | 8.3 | 9.1 | 40 | 48 | 18 |
| 18 | 22.4 | 17.2 | 10.2 | 8.8 | 6.7 | 65 | — | — |
| Mean ± SEM | 20.3 ± 1.1 | 17.3 ± 1.0 | 14.0 ± 0.9 | 11.3 ± 0.8 | 10.1 ± 0.7* | 72 ± 12* | 106 ± 14* | 50 ± 8* |

(*p < 0.05 for mean values group 2 versus group 1)

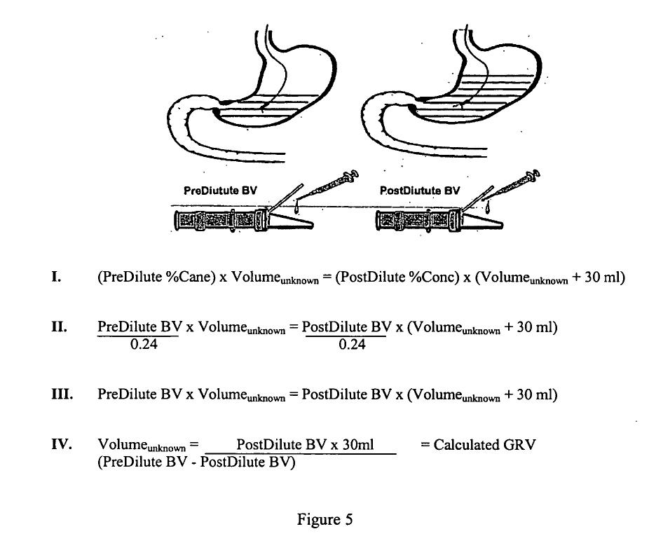

However, conventional use of GRV obtained by aspiration via a syringe may be inaccurate and unreliable in measuring true volume of contents present in the stomach at any given time. The dilution technique (determining BVs before and after addition of a known volume of water, e.g., 30 ml of distilled water) takes advantage of the relationship between the % Concentration of formula at any dilution and the measured BVs shown in FIG. 2, and may be used to calculate the true volume of contents and the specific volume of formula remaining in the stomach. As shown in FIG. 5, it is empiric that while an absolute amount of formula in the stomach does not change with the added dilutional 30 ml volume of distilled water, the total volume of gastric contents increases while the % concentration of formula and the corresponding BV decreases. The original volume of gastric contents present in the stomach before dilution (volume unknown) was derived from the change in the BV following dilution (FIG. 5). The amount of formula remains constant through dilution and is described by equation (1) in FIG. 5. Substituting the product BV/0.24 for % Concentration generates equation (2), both sides of which may be multiplied by 0.24 to derive equation (3). Solving for the volume unknown volume produces equation (4) and provides a value corresponding to the Calculated GRV.

The mean calculated gastric residual volume was shown to be significantly higher for patients in group 2 than for those in group 1 (106±14 versus 67±15 mL, respectively, p<0.05) (Tables 6 and 7). Using the final pre-diluted BV (i.e., the Brix value before water dilution) to derive the % concentration of the dietary formula, in combination with the calculated gastric residual volume (Calc. GRV), the specific volume of dietary formula of dietary present at 180 minutes was determined for both groups. The volume of dietary formula remaining was significantly higher for patients in group 2 compared to those in group 1 (50±8 versus 6±2 mL, respectively, p<0.05) (Tables 6 and 7).

As shown in Tables 6 and 7, use of refractometry in combination with conventional calculation of gastric residual volume identified 4% (1/25) of patients in group 1 with low gastric residual volumes who might have possible gastric dysmotility (>20% of the initial 250 mL bolus volume of formula remaining at 180 minutes). Use of refractometry together with conventional measurement of gastric residual volumes indicated that 72% (13/18) of patients in group 2 with higher gastric residual volumes had sufficient gastric emptying (<20% of initial 250 mL volume of formula remaining).

The full strength dietary formula is rich in dissolved nutrients and displays a high BV of 23.2±0.3. By contrast, saliva and gastric secretions have few dissolved substances and therefore have correspondingly low BVs, close to that of 0.9% sodium chloride (Table 1). By evaluating the BV value, one may determine whether gastric contents are comprised predominately of dietary formula or digestive secretions in patients receiving dietary formula.

Monitoring GRV and Dietary Formula Concentration in Determining Feeding Tolerance

Brix value measurements and monitoring of GRVs and dietary formula concentrations are useful in evaluating feeding tolerance and gastric emptying. Embodying monitoring methods are exemplified in Table 8. A patient carrying a low GRV associated with low food retention (low Brix reading) would be interpreted to indicate that the formula is being emptied appropriately, and that there is no retention within the stomach. The risk for aspiration in the presence of low GRVs would be expected to be minimal, and the patient would be perceived as tolerating feeds. On the other hand, high GRV associated with high food retention would indicate the presence of delayed gastric emptying, an increased volume of gastric contents as a result of formula being retained in the stomach, and true feeding intolerance. However, patients carrying a low GRV but showing evidence of high food retention might be interpreted as having evidence of gastric dysmotility. In that case, the low GRV might represent a false negative screening monitor caused by the fact that the tip of the feeding tube is not in the pool of gastric juice, or the tip of feeding tube is adherent to the gastric mucosa. Low food retention by several BV determinations in a patient with high GRVs, may give some assurance that formula is being emptied effectively from the stomach. The high GRVs would thus represent a false positive screening monitor. Feeds could be continued as close clinical assessment is continued.

TABLE 8
Monitoring food content and residual volume simultaneously (prior to next
feeding) in patients receiving bolus nasogastric feeding.

| Residual | Formula | | |
|---|---|---|---|
| volume | Concentration | | Recommendation for clinical |
| (GRV) | (Brix Value) | Interpretation | practice |
| Low | Low | Good gastric emptying | Continue or even increase the |
| | | | tube feeding |
| High | High | Delayed gastric emptying | Stop or decrease the tube |
| | | | feeding, change from bolus-to |
| | | | continuous feeding, and/or |
| | | | switch to small bowel tube |
| | | | feeding |
| Low | High | May represent gastric | Check the true residual volume |
| | | dysmotility; aspirated | by water dilution technique, |
| | | GRV may be insensitive | close clinical monitoring to |
| | | to true GRV | assure tolerance |
| High | Low | May represent normal | Feeds could be continued with |
| | | emptying of formula; | close assessment of tolerance to |
| | | elevated aspirated GRV | enteral feeding; consider trial of |
| | | may reflect volume of | anti-secretory agents such as |
| | | endogenous secretions | proton pump inhibitor |

### Example 3

### Continuous Nasogastric Tube Feeding: Monitoring by Brix Value and Conventional Gastric Residual Volumes

Materials and Methods

After monitoring for 24 hours, 36 patients on continuous enteral tube feeding with a full strength (100%) polymeric dietary formula (Osmolite HN) were entered in this study and divided into 2 groups based on their pattern of conventional aspirated gastric residual volumes over the monitoring period. Patients with lower aspirated gastric residual volumes (<75 mL) were placed in Group 1, while patients with higher aspirated gastric residual volumes (>75 mL on at least 2 occasions) were placed in Group 2. Aspirated gastric residual volumes were obtained by aspiration of the feeding tube using a 60-ml syringe, first in the supine position, and then in the right lateral decubitus position. Upon entry, all gastric contents were aspirated, the volume recorded (aspirated gastric residual volume), Brix value measurements by refractometry performed, and the contents reinstilled but diluted with 30 mL additional water. Then a small amount was reaspirated, and repeat Brix value measurements were made. Three hours later, the entire procedure was repeated a second time.

The Brix values were measured using a hand-held refractometer, whose Brix scale of 0-32 could be read in 0.2 increments. The refractometer was calibrated with distilled water before each measurement. One or two drops of the specimen fluid were placed on a designated window for observation using daylight at room temperature. In this way, the concentration of soluble solids in solution was measured at the bedside for each specimen.

Results

Results are presented as mean values±SEM. The Student's t test was used to assess differences in results between patients in Group 1 and Group 2. A p value of less than 0.05 was considered to be statistically significant.

No patient in either group demonstrated nausea, vomiting, aspiration, or evidence of clear intolerance of enteral tube feeding. Table 9 shows the raw data of GRVs for the patients in Group 1. Patients in Group 1, with lower Asp GRVs based on the pre-study period of monitoring, continued to demonstrate very low Asp GRVs following entry into the study, with 93% (43/44) of the Asp GRVs obtained on the first and second measurements being <75 ml. In contrast, patients in Group 2 (again differentiated by higher GRVs on pre-study monitoring) continued to show higher Asp GRVs following entry into the study, with only 11% (3/28) of Asp GRVs on both measurements <75 ml (Table 10). Mean aspirated GRV was significantly higher for those patients in Group 2 compared to those in Group 1 on both first (124±7 versus 14±2 ml, respectively, p<0.05) and second (75±10 versus 15±4 ml, respectively, p<0.05) measurements.

TABLE 9
GRVs for Group 1 patients (low residual volume) receiving continuous tube feeding

| | | First measurement (ml) | | | Second measurement (ml) | | |
|---|---|---|---|---|---|---|---|
| Patient | Infusion rate | | Cal | Formula | Asp | | Formula |
| no. | (ml/hr) | Asp GRV | GRV | remaining | GRV | Cal GRV | remaining |
| 1 | 75 | 45 | 78 | 39 | 6 | 35 | 33 |
| 2 | 30 | 40 | 40 | 12 | 15 | 56 | 24 |
| 3 | 75 | 30 | 55 | 24 | 3 | 79 | 72 |
| 4 | 75 | 25 | 74 | 17 | 85 | 90 | 14 |
| 5 | 65 | 20 | 35 | 24 | 4 | 38 | 35 |
| 6 | 20 | 15 | 75 | 6 | 20 | 60 | 9 |
| 7 | 45 | 12 | 33 | 32 | 20 | 59 | 35 |
| 8 | 60 | 10 | 32 | 28 | 5 | 35 | 32 |
| 9 | 75 | 10 | 34 | 32 | 5 | 37 | 31 |
| 10 | 50 | 10 | 31 | 23 | 4 | 37 | 28 |
| 11 | 30 | 10 | 37 | 32 | 35 | 66 | 23 |
| 12 | 55 | 10 | 37 | 35 | 12 | 35 | 32 |
| 13 | 30 | 10 | 31 | 26 | 5 | 35 | 29 |
| 14 | 45 | 8 | 32 | 30 | 3 | 32 | 30 |
| 15 | 40 | 8 | 34 | 31 | 20 | 97 | 23 |
| 16 | 30 | 7 | 31 | 29 | 5 | 32 | 30 |
| 17 | 70 | 7 | 40 | 40 | 11 | 49 | 36 |
| 18 | 70 | 7 | 36 | 36 | 32 | 69 | 32 |
| 19 | 70 | 7 | 40 | 40 | 11 | 49 | 36 |
| 20 | 70 | 6 | 33 | 31 | 5 | 33 | 30 |
| 21 | 60 | 6 | 33 | 31 | 10 | 33 | 31 |
| 22 | 30 | 10 | 34 | 31 | 3 | 39 | 34 |
| Mean ± SEM | 53 ± 4 | 14 ± 2* | 41 ± 3* | 29 ± 2* | 15 ± 4* | 50 ± 4* | 31 ± 2 |

Legend: GRV = gastric residual volume,
Asp GRV = aspirated gastric residual volume,
Cal GRV = calculated gastric residual volume
(*p < 0.05 for mean value Group 1 versus Group 2).

TABLE 10
GRVs for Group 2 patients (high-residual volume) receiving continuous tube feeding

| | | First measurement (ml) | | | Second measurement (ml) | | |
|---|---|---|---|---|---|---|---|
| Patient | Infusion rate | Asp | | Formula | Asp | | Formula |
| no. | (ml/hr) | GRV | Cal GRV | remaining | GRV | Cal GRV | remaining |
| 1 | 40 | 145 | 104 | 19 | 18 | 39 | 37 |
| 2 | 70 | 142 | 130 | 51 | 110 | 146 | 62 |
| 3 | 75 | 140 | 171 | 65 | 110 | 149 | 71 |
| 4 | 40 | 110 | 200 | 33 | 10 | 37 | 34 |
| 5 | 40 | 98 | 88 | 18 | 9 | 43 | 39 |
| 6 | 30 | 128 | 138 | 135 | 97 | 62 | 27 |
| 7 | 35 | 120 | 116 | 114 | 95 | 50 | 26 |
| 8 | 30 | 110 | 140 | 141 | 85 | 47 | 22 |
| 9 | 30 | 160 | 150 | 87 | 80 | 132 | 62 |
| 10 | 40 | 155 | 110 | 69 | 90 | 115 | 61 |
| 11 | 30 | 138 | 132 | 75 | 86 | 117 | 51 |
| 12 | 70 | 130 | 180 | 72 | 100 | 180 | 77 |
| 13 | 30 | 85 | 193 | 72 | 78 | 119 | 47 |
| 14 | 30 | 78 | 282 | 110 | 80 | 193 | 72 |
| Mean ± SEM | 42 ± 4 | 124 ± 7* | 152 ± 13* | 76 ± 10* | 75 ± 10* | 102 ± 15* | 49 ± 5* |

Legend: GRV = gastric residual volume,
Asp GRV = aspirated gastric residual volume,
Cal GRV = calculated gastric residual volume
(*p < 0.05 for mean value Group 1 versus Group 2).

Tables 11 and 12 show the pattern of the first and second Brix value measurements pre- and post-dilution. In general, patients in Group I tended to show of pattern of high pre-dilution Brix values dropping further to lower post-dilution Brix values than those patients in Group 2 (which showed the opposite pattern low pre-dilution Brix values dropping to a less extent to higher post-dilution Brix values). Only the difference in post-dilution Brix values between the two groups on both measurements reached statistical significance.

This pattern in Group 1 patients suggested that gastric contents were comprised of enteral formula of fairly high concentration (as evidenced by pre-dilution Brix values close to the in vitro Brix values of 23.2 for full strength Osmolite HN) that was of very small volume (as evidenced by the tremendous drop in Brix values with dilution by a small 30 ml volume of distilled water). The opposite pattern in Group 2 patients, in contrast, suggested greater dilution by endogenous secretions (as evidenced by pre-dilution Brix values less than the Brix value of 23.2 for full strength Osmolite HIS and greater total volume (as evidenced by the lesser drop in Brix values with dilution by a small 30 ml volume of distilled water).

The mean Brix value ratios for the two groups reflected these distinct patterns. The mean Brix value ratio was significantly higher for those patients in Group 2 compared to those in Group 1 on both the first (79±2% versus 20 t 4%, respectively, p<0.05) and second (59±7% versus 32±5%, respectively, p<0.05) measurements. For those patients in Group I, Brix value ratios on all measurements for all patients were <70% (Table 11). For those patients in Group 2, the Brix value ratios on the first measurement were >70% on all but one patient (patient #5) (Table 12). When measured the second time, 6 of the 14 patients (patients #1, 4-8) showed that the Brix value ratios had fallen to <70%.

TABLE 11
Brix values for Group 1 patients (low residual volume) receiving continuous tube feeding

| | First measurement (ml) | | | Second measurement (ml) | | |
|---|---|---|---|---|---|---|
| Patient | Pre-dilution | Post-dilution | Brix value | Pre-dilution | Post-dilution | Brix value |
| no. | Brix value | Brix value | ratio | Brix value | Brix value | ratio |
| 1 | 12.0 | 7.4 | 62% | 23.0 | 3.0 | 13% |
| 2 | 7.4 | 1.8 | 24% | 10.4 | 4.8 | 46% |
| 3 | 10.6 | 4.8 | 45% | 22.0 | 13.6 | 62% |
| 4 | 5.4 | 3.2 | 59% | 3.6 | 2.4 | 67% |
| 5 | 16.0 | 2.4 | 15% | 22.6 | 4.6 | 20% |
| 6 | 2.0 | 1.2 | 60% | 3.6 | 1.8 | 50% |
| 7 | 23.4 | 2.0 | 9% | 14.2 | 7.0 | 49% |
| 8 | 21.0 | 1.6 | 8% | 22.2 | 3.0 | 14% |
| 9 | 22.6 | 2.4 | 11% | 20.2 | 3.6 | 18% |
| 10 | 18.0 | 0.4 | 2% | 18.0 | 3.4 | 19% |
| 11 | 21.0 | 4.0 | 19% | 8.4 | 4.6 | 55% |
| 12 | 22.6 | 4.2 | 19% | 22.0 | 3.0 | 14% |
| 13 | 20.0 | 0.6 | 3% | 20.0 | 3.0 | 15% |
| 14 | 22.8 | 1.4 | 6% | 22.6 | 1.4 | 6% |
| 15 | 22.0 | 2.4 | 11% | 5.8 | 4.0 | 69% |
| 16 | 22.2 | 1.0 | 5% | 22.6 | 1.2 | 5% |
| 17 | 23.8 | 6.0 | 25% | 17.6 | 6.8 | 39% |
| 18 | 23.6 | 4.0 | 17% | 11.0 | 6.2 | 56% |
| 19 | 23.8 | 6.0 | 25% | 19.6 | 6.8 | 39% |
| 20 | 22.4 | 2.0 | 9% | 22.0 | 1.8 | 8% |
| 21 | 23.0 | 2.0 | 9% | 22.2 | 2.2 | 10% |
| 22 | 22.0 | 2.6 | 12% | 20.8 | 4.8 | 23% |
| Mean ± SEM | 18.5 ± 1.4 | 2.9 ± 0.4* | 20 ± 4%* | 16.9 ± 1.4 | 4.2 ± 0.6* | 32 ± 5%* |

*p < 0.05 for mean value Group 1 versus Group 2

TABLE 12
Brix values for Group 2 patients (high residual volume) receiving continuous tube feeding

| | First measurement (ml) | | | Second measurement (ml) | | |
|---|---|---|---|---|---|---|
| Patient | Pre-dilution | Post-dilution | Brix value | Pre-dilution | Post-dilution | Brix value |
| no. | Brix value | Brix value | ratio | Brix value | Brix value | ratio |
| 1 | 4.5 | 3.2 | 71% | 22.7 | 5.4 | 24% |
| 2 | 9.5 | 7.3 | 77% | 10.2 | 8.1 | 79% |
| 3 | 9.1 | 7.5 | 82% | 11.4 | 9.1 | 80% |
| 4 | 4.0 | 3.4 | 85% | 22.3 | 4.0 | 18% |
| 5 | 5.0 | 3.3 | 66% | 22.0 | 6.5 | 30% |
| 6 | 23.5 | 18.4 | 78% | 10.4 | 5.4 | 52% |
| 7 | 23.6 | 17.5 | 74% | 12.4 | 5.0 | 40% |
| 8 | 24.2 | 19.0 | 79% | 11.0 | 4.0 | 36% |
| 9 | 14.0 | 11.2 | 80% | 11.4 | 8.8 | 77% |
| 10 | 15.0 | 10.9 | 73% | 12.7 | 9.4 | 74% |
| 11 | 13.6 | 10.5 | 77% | 10.5 | 7.8 | 74% |
| 12 | 9.6 | 8.0 | 83% | 10.2 | 8.5 | 83% |
| 13 | 9.0 | 7.6 | 84% | 9.5 | 7.1 | 75% |
| 14 | 9.4 | 8.4 | 89% | 9.0 | 7.6 | 84% |
| Mean ± SEM | 12.4 ± 1.9 | 9.7 ± 1.4* | 79 ± 2%* | 13.3 ± 1.3 | 6.9 ± 0.5* | 59 ± 7%* |

*p < 0.05 for mean value Group 2 versus Group 1

As a whole, patients in Group 1 had a volume of formula estimated to be remaining on both measurements that was very low, reinforcing the pattern shown by Brix value ratios. (Table 9). In one patient (patient #22) where the volume of formula was greater than the hourly feeding rate, aspirated GRV and calculated GRV were <40 ml. Overall, 95% (21/22) of the Group 1 patients had a volume of formula remaining on both measurements that was less than the hourly feeding rate.

In contrast to those patients in Group 1, patients in Group 2 showed evidence of reduced gastric emptying and greater volume of retained formula (Table 10). Comparing the formula estimated to be remaining with the hourly infusion rate, six patients (patients #9-14) showed concurrence on both measurements for evidence of retention of formula and decreased or impaired gastric emptying. For these six patients, the volume of formula remaining was estimated to be greater than the hourly infusion rate on both measurements. In these six, both the first and second Brix value ratios were >70%. Traditional Asp GRV, however, was insensitive and failed to identify these patients, being [100 ml on two of the six first round of measurements, and six out of six on the second round of measurements. Three patients from Group 2 (patients #6-8), who on initial measurement showed retention of formula, improved on follow-up measurement indicating adequate gastric emptying of the formula. Five patients could be identified (patients #1-5) with high Asp GRVs from Group 2 who had a volume of formula remaining estimated to be less than the hourly infusion rate, assuring adequate gastric emptying all along.

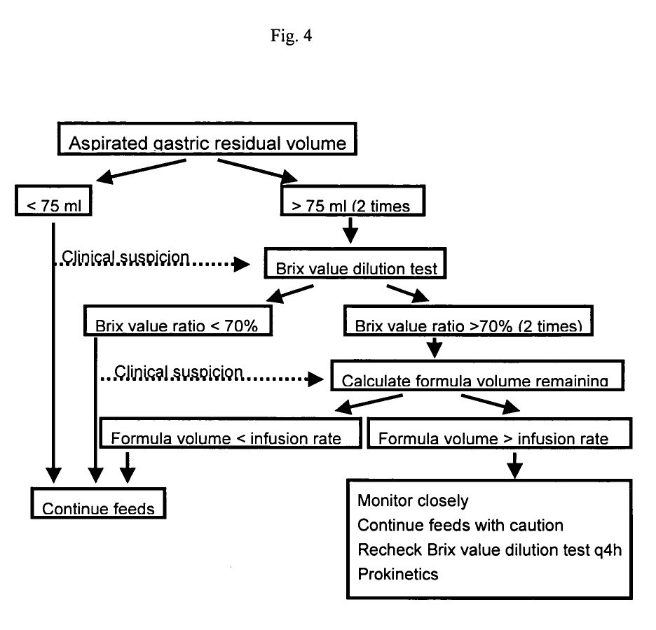

FIG. 4 and the results of this study demonstrate how refractometry may be used in conjunction with conventional measurement of aspirated gastric residual volumes to create a strategy by which to monitor patients on continuous gastric feeding. As shown in this study, patients who continue to have lower aspirated gastric residual volumes (for example, less than 75 mL) appear to be at low risk, and should have good tolerance and sufficient gastric emptying, and do not require refractometry assessment (unless clinical suspicion of intolerance increases, based on abdominal distension, reduced passage of gas and stool, increasing nausea, or regurgitation and vomiting). If patients are found to have higher aspirated gastric residual volumes (for example, greater than 75 mL on more than two occasions) then a Brix value dilution test is performed. Of course, the ordinary clinician or practitioner is skilled in determining whether the aspirated gastric residual volume is relatively higher or lower, dependent upon the clinical setting of the subject (patient).

As noted elsewhere in this application, a Brix value dilution test may be performed by measuring the Brix value of a gastric content sample to obtain a pre-dilution Brix value, adding a known volume of water to the gastric content sample to form a post-dilution sample, infusing the post-dilution sample into the stomach of a subject (patient), aspirating the post-dilution sample from the stomach, measuring the Brix value of the aspirated post-dilution sample to obtain a post-dilution Brix value, and dividing the post-dilution Brix value by the pre-dilution Brix value to obtain a Brix value ratio.

In one embodiment, the Brix value ratio is less than 70%, as this study has shown, there is a low likelihood for retention of formula and feeds may continue. In another embodiment, if the Brix value ratio is greater than 70% on for example, more than 2 occasions (e.g., performed every 4 hours), the clinician calculates the volume of dietary formula remaining in the stomach. The number of times that the Brix value ratio is greater than 70% and the time interval between performing successive tests is dependent on the clinical setting of the patient and easily determined by the ordinary practitioner. The finding that the volume of dietary formula remaining in the stomach is greater than the hourly infusion rate alerts the clinician to the possibility of delayed gastric emptying with retention of formula, and that feeds may be continued with caution under close observation (for example, continuing to perform Brix value dilution tests with aspirated gastric residual volumes every 4 hours or some other determined time period).

### Example 4

### Determination of Gastric Residual Volume (GRV)

Materials and Methods

Forty-three patients receiving bolus nasogastric feeding were monitored for 24 hours prior to entry into the study, and then divided into two groups based on conventional use of GRV; patients with low GRVs (<75 ml) were placed in group 1, while patients with higher GRVs (>75 ml) were placed in group 2. All subjects were given 250 ml of polymeric formula by bolus nasogastric infusion, followed by Brix value (BV) measurement of gastric contents at 0, 30, 60, 120, and 180 minutes. All gastric fluid was aspirated after 180 minutes of feeding; the volume was recorded (Aspirate GRV) and BV made (PreDilute BV), then reinstilled with an added 30 ml of dilutional water, after which a final aspiration and BV measurement (PostDilute BV) was performed. Calculated GRV was determined by the equation: Calculated GRV×PostDilute BV=(Calculated GRV +30 ml)×PostDilute BV Specific volume of formula at 180 minutes was defined by 2 equations; % Concentration=BV_{180 min}/0.24 and Volume_{formula}=% Concentration×Calculated GRV.

Results

Serial BV measurements decreased in both groups after bolus feeding. For patients in group 2 the decrease was less, such that at 180 minutes the mean BV for gastric contents was, significantly higher than for those patients in group 1 (10.1 versus 5.1, respectively, p<0.01). Aspirate GRV, Calculated GRV, and Volume_{formula} present at 180 minutes was significantly greater for patients in group 2 compared to those in group 1. Use of refractometry in combination with traditional use of GRV identified 4% (1/25) of patients in group 1 with low GRVs who might have possible gastric dysmotility (>20% of initial 250 ml volume of formula remaining at 180 minutes), and assured that 72% (13/18) patients in group 2 with higher GRVs had sufficient gastric emptying (<20% of initial 250 ml volume of formula remaining).

Throughout this specification, unless the context requires otherwise, the word “comprise,” or variation such as “comprises” or “comprising,” will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present application before the priority date of each claim of this application before the priority date of each claim of this application.

It will be appreciated by persons skilled in the art that numerous variations and/or modifications may be made to the embodiments without departing from the spirit or scope of the claims as broadly described. Equivalents for the particular embodiments discussed in this description may practice the claims as well. The present embodiments are, therefore, to be considered in all respects as illustrative and not restrictive.

What is claimed is:

1. A method of determining the concentration of dietary formula, comprising:
infusing dietary formula into the stomach of a subject;
measuring a Brix value of the infused dietary formula;
determining a slope value derived from Brix values of serially diluted dietary formula over a determined concentration range; and
dividing the Brix value of the infused dietary formula by the slope value.

2. The method according to claim 1, whereby the slope value is 0.24.

3. The method according to claim 1, whereby the dietary formula is a formula for enteral nutrition, a formula for gastric tube feeding, liquid dietary formula, or polymeric diet formula.

4. The method according to claim 1, whereby the concentration exhibits a linear relationship to the Brix value of the infused dietary formula.

5. The method according to claim 1, whereby the serially diluted dietary formula is diluted in distilled water, gastric juice, saliva, dextrose solution, or sodium chloride solution.

6. A method of determining gastric residual volume, comprising:
(a) measuring the Brix value of a gastric content sample to obtain a pre-dilution Brix value;
(b) adding a known volume of water to the gastric content sample to form a post-dilution sample;
(c) infusing the post-dilution sample into the stomach of a subject;
(d) aspirating the post-dilution sample from the stomach;
(e) measuring the Brix value of the aspirated post-dilution sample to obtain a post-dilution Brix value; and
(f) multiplying the Brix value of the gastric content sample by the known volume of water and dividing the resulting product by the difference between pre-dilution Brix value and the post-dilution value.

7. The method according to claim 6, whereby step (a) is obtained from an in vitro sample or a sample aspirated from a stomach infused with dietary formula.

8. The method according to claim 6 whereby the gastric content sample is aspirated from the stomach of a patient.

9. The method according to claim 6, whereby at least one Brix value is measured by refractometer.

10. A method of measuring the volume of dietary formula remaining in stomach, comprising:
calculating the gastric residual volume of a subject; and
multiplying the calculated gastric residual volume by the percent concentration of dietary formula infused in the stomach.

11. The method according to claim 10, whereby the dietary formula is the formula for enteral nutrition, a formula for gastric tube feeding, liquid dietary formula or polymeric diet formula.

12. A method of measuring gastric juice volume in stomach, comprising:
measuring the volume of dietary formula remaining in the stomach of a subject;
calculating or measuring the gastric residual volume; and
determining the value derived from the difference between the calculated gastric residual volume and the dietary formula volume remaining in the stomach.

13. The method according to claim 12, whereby the volume of dietary formula remaining in stomach is calculated by determining the gastric residual volume of a subject and multiplying the gastric residual volume by the percent concentration of dietary formula infused in the stomach.

14. The method according to claim 12, whereby the gastric residual volume is calculated by:
(a) measuring the Brix value of a gastric content sample to obtain a pre-dilution Brix value;
(b) adding a known volume of water to the gastric content sample to form a post-dilution sample;
(c ) infusing the post-dilution sample into the stomach of a subject;
(d) aspirating the post-dilution sample from the stomach;
(e) measuring the Brix value of the aspirated post-dilution sample to obtain a post-dilution Brix value; and
(f) multiplying the Brix value of the gastric content sample by the known volume of water and dividing the resulting product by the difference between pre-dilution Brix value and the post-dilution value.

15. The method according to claim 14, whereby the dietary formula is a formula for enteral nutrition, a formula for gastric tube feeding, liquid dietary formula, or polymeric diet formula.

16. A method of monitoring gastric content emptying and feeding tolerance in a patient receiving dietary formula, comprising:
infusing dietary formula into the stomach of a patient;
calculating a gastric residual volume;
aspirating the gastric contents of the patient;
measuring the Brix value of the aspirated gastric contents to obtain a pre-dilution Brix value;
calculating the gastric residual volume;
reinstilling the gastric contents along with a known volume of water;
reaspirating at least a portion of the sample and measuring its Brix value to obtain a post-dilution Brix value; and
calculating the volume of contents remaining in stomach by multiplying the known volume of water by the pre-dilution Brix value and dividing the resulting product by the difference between the pre-dilution Brix value and the post-dilution Brix value.

17. A method of determining the volume of dietary formula remaining in stomach, comprising:
calculating a gastric residual volume of the patient infused with dietary formula;
measuring the Brix value of an aspirated sample from the infused stomach; and
multiplying the gastric residual volume by the Brix value of the aspirated sample and dividing the resulting product by 0.24.

18. A method of monitoring feeding tolerance in a patient receiving enteral nutrition, comprising:
infusing dietary formula into the stomach of a patient;
measuring the aspirated gastric residual volume of an infused patient;
evaluating the aspirated gastric residual volume, whereby a lower gastric residual volume value indicates acceptable feeding tolerance and a higher gastric residual volume value indicates additional monitoring;
calculating a Brix value ratio obtained by a Brix value dilution test;
evaluating the Brix value ratio, whereby a ratio of less than approximately 70% indicates acceptable feeding tolerance and a ratio above approximately 70% indicates additional monitoring;
calculating the formula volume remaining in the stomach; and
evaluating the volume of formula remaining in the stomach, whereby a formula volume approximately less than the infusion rate indicates acceptable feeding tolerance and a formula volume greater than the infusion rate indicates additional monitoring.

19. The method according to claim 18, where by the lower gastric residual volume is less than approximately 75 mL and the higher gastric residual volume value is above approximately 75 mL.

20. The method according to claim 18, whereby the gastric residual volume is calculated by:
(a) measuring the Brix value of a gastric content sample to obtain a pre-dilution Brix value;
(b) adding a known volume of water to the gastric content sample to form a post-dilution sample;
(c) infusing the post-dilution sample into the stomach of a subject;
(d) aspirating the post-dilution sample from the stomach;
(e) measuring the Brix value of the aspirated post-dilution sample to obtain a post-dilution Brix value; and
(f) multiplying the Brix value of the gastric content sample by the known volume of water and dividing the resulting product by the difference between pre-dilution Brix value and the post-dilution value.

21. The method according to claim 18, whereby the Brix value ratio is calculated by dividing the pre-dilution Brix value by the post-dilution Brix value.

22. The method according to claim 18, whereby the Brix value dilution test further comprises:
measuring the Brix value of a gastric content sample to obtain a pre-dilution Brix value;
adding a known volume of water to the gastric content sample to form a post-dilution sample;
infusing the post-dilution sample into the stomach of a subject;
aspirating the post-dilution sample from the stomach;
measuring the Brix value of the aspirated post-dilution sample to obtain a post-dilution Brix value; and
dividing the post-dilution Brix value by the pre-dilution Brix value to obtain a Brix value ratio.

23. The method according to claim 18, whereby the volume of formula remaining in the stomach is calculated by determining the gastric residual volume of a subject and multiplying the gastric residual volume by the percent concentration of dietary formula infused in the stomach.

24. The method according to claim 18, whereby the volume of formula remaining in the stomach is calculated by:
calculating a gastric residual volume of the patient infused with dietary formula;
measuring the Brix value of an aspirated sample from the infused stomach; and
multiplying the gastric residual volume by the Brix value of the aspirated sample and dividing the resulting product by 0.24.
